(19) United States
(12) Patent Application Publication
(10) Pub. No.: US 2007/0026428 A1
(43) Pub. Date: Feb. 1, 2007

(54) Combinatorial expression of split caspase molecules

(76) Inventor: Dattananda S. Chelur, New York, NY (US)

Correspondence Address: BAKER & BOTTS L.L.P., 30 ROCKEFELLER PLAZA, 44TH FLOOR, NEW YORK, NY, 10112-4498, US

(21) Appl. No.: 11/416,845

(22) Filed: May 2, 2006

Related U.S. Application Data
(60) Provisional application No. 60/677,131, filed on May 2, 2005.

(51) Int. Cl. C12Q 1/68; A01K 67/033; C07H 21/04; C12P 21/06; C12N 9/64

(52) U.S. Cl. 435/6; 435/69.1; 435/226; 435/320.1; 435/325; 536/23.2; 800/8

(57) ABSTRACT

The present invention relates to the use of split caspase proteins to determine whether or not promoters are coordinately active, whereby the transcriptional expression of incomplete portions of a caspase protein is controlled by different promoters and coordinate (not necessarily contemporaneous) promoter activity results in formation of an activated caspase protein and, consequently, apoptotic cell death. The present invention further provides for the use of an additional promoter element controlling expression of a “caspase neutralizing protein,” which, when present, inhibits the apoptotic effect of the assembled caspase subunits. Rescue of cells that actively transcribe the complementary caspase subunits indicates that all promoters of the system are coordinately active. The present invention, in non-limiting embodiments, may be used to selectively ablate cells in the context of cultures as well as intact organisms, and provides means of demonstrating coordinate activity of multiple promoters.

### PRIORITY CLAIM

This application claims priority to U.S. Provisional Patent Application Ser. No. 60/677,131, filed May 2, 2005, the contents of which is hereby incorporated in its entirety herein.

### GRANT INFORMATION

The subject matter of this application was developed at least in part under National Institutes of Health Grant No. GM30997, so that the United States Government has certain rights herein.

### 1. INTRODUCTION

The present invention relates to the use of split caspase, proteins to determine whether or not promoters are coordinately active, whereby the transcriptional expression of incomplete portions of a caspase protein is controlled by different promoters and coordinate (not necessarily contemporaneous) promoter activity results in formation of an activated caspase protein and, consequently, apoptotic cell death. The present invention further provides for the use of an additional promoter element controlling expression of a “caspase neutralizing protein,” which, when present, inhibits the apoptotic effect of the assembled caspase subunits. Rescue of cells that actively transcribe the complementary caspase subunits indicates that all promoters of the system are coordinately active. The present invention, in non-limiting embodiments, may be used to selectively ablate cells in the context of cultures as well as intact organisms, and provides means of demonstrating coordinate activity of multiple promoters.

### 2. BACKGROUND OF THE INVENTION

### 2.1 Targeted Cell Killing

A frequent goal in both clinical medicine as well as scientific research is to selectively eliminate one specific type of cell. In cancer therapy, clinicians strive to kill the cancer cells without damaging the healthy cells of their patients. In research, scientists often ask the question, “what does this type of cell do?” by seeing what happens when that class of cells is destroyed. In each case, achieving targeted cell death has, historically, been a problem because, in addition to the distinguishing features between cell types, there is much commonality. Many chemotherapeutic agents target proliferating cells, because most cancer cells rapidly divide; unfortunately, so do normal cells in the bone marrow, so that cancer patients undergoing chemotherapy suffer temporary damage to their immune systems.

With the advent of recombinant DNA technology and advances in molecular biology, it became possible to target the production of a bioactive molecule to a particular class of cells by using a cell type specific promoter. Thus, a cancer-suppressing protein could be selectively expressed in cancer cells by putting the gene encoding that protein under the control of a promoter that is selectively active in cancer cells. This approach, while somewhat successful, is not without its problems. Very few promoters are active in only one type of cell-frequently there is a certain baseline level of activity outside the target cell population. Furthermore, the number of cell-type specific promoters known is limited, and there are certain types of cells for which no rigorously specific promoter is available. Therefore, it is desirable to develop means of selectively targeting a specific cell population which have minimal or no effect on other cell types.

### 2.2 Caspases

Cysteine proteases are defined as peptidases (protein cleaving enzymes) that have a cysteine residue at their catalytically active center. A group of cysteine proteases, the cysteinyl aspartate-specific proteases, or so-called “Caspases,” encompasses cysteine proteases that in addition possess a strict requirement for cleaving their substrates after an aspartic acid residue.

Historically, the existence of a caspase was first reported in 1992 as an enzyme responsible for proteolytic processing of interleukin-1β, named ICE (interleukin-1β converting enzyme; Thornberry et al., 1992, Nature 356:768-774; Cerretti et al., 1992, Science 256:97-100). Recognition that ICE possesses homology to the product (CED-3) of the nematode Caenorhabditis elegans ced-3 gene, which is involved in apoptotic cell death (Yuan et al., 1993, Cell 75:641-652), has lead to abundant research and a better understanding of the mechanisms of apoptosis in higher organisms. Evolutionarily, caspases are found strictly in metazoan animals with distant homologs present in plants and bacteria (Funtes-Prior and Salvesen, 2004, Biochem. J. 384:201-232).

At least 11 members of the human caspase family have been reported in the literature (Id.). Among these, seven participate in the initiation and execution of apoptosis or programmed cell death. Three, in particular caspase-1 and probably caspases-4 and -5, are involved in production of the proinflammatory cytokines (Id.) and one, caspase-14, is found mainly in the epidermis and may be involved in keratinocyte differentiation.

Caspases share a number of common features, including (i) synthesis as catalytically inactive zymogens, (ii) activation by cleavage of a specific internal aspartic acid to form a small and a large subunit, which associate to form the biologically active molecule, and (iii) specific cleavage of substrate after an aspartic acid residue. Certain mature active caspases, in particular those that possess long prodomains, can process and activate their own and other inactive caspase zymogens (Fernandez-Alnemri et al., 1996, Proc. Natl. Acad. Sci. U.S.A. 93:7464-7469). This activation process is sequential, usually specific, and determined by the caspase preference toward the target P4-P1 subsite, which is present in the interdomain linker between a large and a small subunit of the caspase zymogen.

Interestingly, the short prodomain caspases contain target sites that are preferred by the long prodomain caspases (Thornberry et al., 1997, J. Biol. Chem. 272:17907-17911). This observation has led to the suggestion that caspases operate in a hierarchical relationship within an intracellular network of proteolytic signaling pathways or cascades (Salvesen and Dixit, 1997, Cell 91:443-446; Cohen, 1997, Biochem. J. 326:1-16). Thus, implementation of the apoptotic program is now believed to require the participation of at least two classes of caspases, the initiator and the executioner caspases.

In mammalian systems three initiator or apical caspases, namely caspases-2, -8, and -10, have been implicated in apoptotic pathways triggered by the death receptors of the tumor necrosis factor receptor family (Funtes-Prior and Salvesen, 2004, Biochem. J. 384:201-232). Upon ligand-induced trimerization of the death receptors, the initiator caspases are recruited through their long N-terminal prodomains by specialized adaptor molecules to form the death-inducing signaling complex (DISC). For example, caspase-8 and probably caspase-10 are recruited to the DISC by the adaptor molecule FADD/Mort1, whereas caspase-2 is recruited by CRADD/RAIDD and RIP (Nagata, 1997, Cell 88:355-365). Because of the trimeric nature of the DISC, three caspase molecules are brought in close proximity to one another, which is believed to facilitate their activation by autocatalytic processing (Muzio et al., 1998, J. Biol. Chem. 273:2926-2930). Caspase-9, another long prodomain initiator caspase, is activated by binding to Apaf-1 (Li et al., 1997, Cell 91:479-489). The exact mechanism by which Apaf-1 triggers activation of caspase-9 has not been defined. However, the release of cytochrome c from mitochondria, a prerequisite for formation of the Apaf-1-caspase-9-cytochrome c complex, is believed to be triggered by many apoptotic stimuli, including those initiated by other initiator caspases (Reed, 1997, Cell 91:559-562). The downstream or executioner caspases, namely caspases-3, -6, and -7, lack long prodomains that are required for recruitment to caspase activation complexes such as the DISC or Apaf-1. These caspases remain dormant until the initiator caspases activate them by direct proteolysis (Li et al., 1997, Cell 91:479-489).

Mutagenesis studies have been performed on the caspases to study structure function relationships (Funtes-Prior and Salvesen, 2004, Biochem. J. 384:201-232). In this context, two recombinant constitutively active caspase-3 and -6 mutants (Srinivasula et al., 1998, J. Biol. Chem. 273(17):10107-10111) have been engineered by switching the order of their two subunits, such that the engineered molecule mimics a structure presented by the processed wild type active molecule. These caspases were designated reversed-caspases-3 and -6 (“rev-caspases-3 and -6”). Unlike their wild type counterparts, the rev-caspases were reported to be capable of autocatalytic processing in an in vitro translation reaction and rapid induction of apoptosis in vivo without proteolytic processing by upstream initiator caspases.

### 2.3 Caspase Neutralizers

Unregulated caspase activity would be lethal, and therefore cells contain additional protective mechanisms to control caspase activity in addition to synthesis and storage of caspases as latent precursors (zymogens or procaspases). Additionally, due to the critical role played by caspases in the immune response, pathogens and in particular viruses have evolved means of inhibiting caspase activity to inhibit immune responses and/or prevent host cell death.

One inhibitory strategy adopted by caspase inhibitors is interruption of the assembly of a functional death inducing signaling complex (DISC) by acting as a decoy molecule which competes with procaspase or other targets and prevents the assembly of a functional DISC. Thus several γ-herpesviruses and tumorigenic molluscipoxvirus block the extrinsic apoptotic induction pathway utilizing decoy molecules (v-FLIP) of this nature (Thome et al., 1997, Nature 386:517-521; Hu et al., 1997, J. Biol. Chem. 272:9621-9624).

Alternatively, a caspase inhibitor may act as an active-site-directed inhibitor, an example of which includes the cowpox protein, CrmA (Gettins, 2002, Chem. Rev. 1 02:4751-4804) which is a member of a superfamily of inhibitors called Serpins. Other examples include the baculovirus p35 protein and a related homolog known as p49 which have no structural similarity or homology to the Serpins though both share a similar mode of inhibition (Jabbour et al., 2002, Cell Death Differ. 9:1311-1320; Zoog et al., 2002, EMBO J. 21:5130-5140). The CrmA and the baculoviral proteins act by serving as substrate decoys of caspases but in addition, after the caspase has acted on the inhibitor by binding to it as it would a normal substrate, kinetic trapping of a reaction intermediate occurs at the active site of the enzyme. In addition, there is a restructuring of inhibitor conformation and/or the cognate caspase as a result of the enzymatic reaction. The end result of the interaction between caspase and inhibitor is an abortive enzymatic reaction that leads to inhibition of the caspase as well as alteration of the inhibitor. Thus, CrmA and the baculoviral proteins p35 and p49 are classified as suicide or mechanism-based inhibitors (Bode and Huber, 2000, Biochim. Biophys. Acta. 1477:241-252).

Another potent inhibitor of caspases was discovered following studies on the apoptotic mechanisms in cells infected with baculoviruses that lacked functional p35 protein. This work led to the discovery of the IAPs (Inhibitor of Apoptosis) family of molecules in baculoviral infected cells as well as by homology in human systems. This family of proteins is characterized by an approximately 70-80 amino acid residue Zn⁺-binding conserved domain called BIR (baculoviral IAP repeat) (Crook et al., 1993, J. Virol. 67:2168-2174). Eight IAPs have been identified in human and current evidence indicates that the endogenous IAPs are likely the most important endogenous control point for apoptosis (Deveraux and Reed, 1999, Genes Dev. 13:239-252).

Numerous studies indicate that caspases are attractive targets for therapeutic intervention in disease states characterized by excessive apoptosis. For example injection of synthetic pan-caspase inhibitors has indicated that decrease of caspase activity is protective in animals (Kreuter et al., 2004, Arch. Immunol. Ther. Exp., 52:141-155; Kawasaki et al., 2000, Am. J. Pathol. 157:597-603) with acute lung injury, nephrotoxic nephritis or myocardial infarction. Typically, the synthetic inhibitors comprise modified tetra- or tri-peptide pseudosubstrates of a caspase cleavage sequence. An alternative strategy for caspase inhibition involves the utilization of small interfering RNA molecules (RNAi) that can be delivered as short double stranded oligonucleotides. RNAi is demonstrated to be highly effective and specific when active and destroys or prevents protein translation of the targeted caspase molecule (Zender et al., 2003, Proc. Natl. Acad. Sci. U.S.A., 98:7797-7802).

### 3. SUMMARY OF THE INVENTION

The present invention relates to reconstitution of caspase activity by coordinately active promoters, whereby the transcriptional expression of incomplete portions of a caspase protein is controlled by different promoters and coordinate (not necessarily contemporaneous) promoter activity results in formation of an activated caspase protein and, consequently, apoptotic cell death. It is based, at least in part, on the discovery that large and small subunits of either CED-3 from C. elegans or Caspase-3 from humans, each linked to a complementary binding partner and placed under the control of separate promoters, produced apoptotic cell death in cells in which both promoters were active.

In further embodiments, the present invention provides for the use of an additional promoter element for controlling expression of a “CAspase NeuTralizer,” (“CANT”) which, when present, inhibits the apoptotic effect of an activated caspase molecule formed either by assembled caspase subunits or by the expression of a reverse caspase. Rescue of cells that would otherwise apoptose demonstrates the coordinate activity of the promoter driving CANT expression and the promoter(s) expressing activated caspase.

The present invention, in non-limiting embodiments, may be used to selectively ablate cells in the context of cultures as well as intact organisms, and provides means of demonstrating coordinate activity of multiple promoters. Further, the requirement of coordinate activity of multiple promoters to assemble activated caspase may be used in therapeutic applications, as it provides a greater ability to target cell death to a specific class of cells.

### 4. BRIEF DESCRIPTION OF THE FIGURES

FIG. 1A-C. Strategy for reconstituting recCED-3 and recCaspase-3. (A) Schematic representation of the proenzyme forms of CED-3 and Caspase-3 is shown. The aspartic acid cleavage sites (D followed by amino acid number), prodomain, large subunit and small subunit boundaries, and mature sizes of large and small subunits are also shown. (B) Schematic representation of fusion constructs comprising leucine zippers CZ and NZ fused to large and small caspase subunits respectively. (C) (from right to left) schematic representation of the caspase leucine zipper fusions showing large and small subunit leucine zippers (sawtooth shaped elements), large and small caspase subunit (cylindrical elements) prior to binding (left panel); binding of the two leucine zipper regions to each other (middle panel); and association of the large and small caspase subunits after leucine zipper association, to generate an active caspase (right panel).

FIG. 2A-C. (A) Percent of GFP positive cells in adult animals transformed with CED-3 subunits, recCED-3 (an activated CED-3), wt-recCED-3, or recCaspase-3. (B) Worm transformed with recCED-3. (C) Worm transformed with recCaspase-3.

FIG. 3A-E. recCaspase-3 activity in HeLa cells. (A) Percent of YFP positive cells localized to the nucleus, in the absence (−) or presence (+) of doxycycline, in HeLa cells transformed with vector, caspase-3 subunits (without leucine zipper), or recCaspase-3 (under the control of a Tet-inducible promoter). (B) Immunofluorescence of YFP in HeLa cells transfected with recCaspase-3 under the control of a Tet-inducible promoter and a caspase-sensing EYFP vector, in the absence of doxycycline. (C) Photomicrograph corresponding to B. (D) Immunofluorescence of YFP, localized to nucleus, in HeLa cells transfected with recCaspase-3 under the control of a Tet-inducible promoter and a caspase sensing EYFP vector, in the presence of doxycycline. (E) Photomicrograph corresponding to D.

FIG. 4A-E. AVD killing by recCED-3 expression from combination of cfi-1 and nmr-1 promoters. (A) Worm transformed with Pₙₘᵣ₋₁yfp. (B) Worm transformed with P_{cji-1}yfp. (C and D) The death of an AVD neuron (arrow) in an animal expressing recCED-3 from Pₙₘᵣ₋₁cz::ced-3(p17) and P_{cfi-1}ced-3(p15)::nz as seen by fluorescence (C) and differential interference contrast (D). This animal also expressed YFP from the Pₙₘᵣ₋₁ promotor. (E) Diagram showing that Pₙₘᵣ₋₁ (left circle) and P_{cfi-1} (right circle) promoters are both active in AVD cells (intersection of sets).

FIG. 5A-E. Restriction of GFP to FLP neurons by recCED-3 mediated cell death. (A and C) Without recCED-3, P_{mec-3}gfp is expressed in the ALM and PLM touch neurons and the FLP neurons of a newly hatched larva: (A) differential interference contrast image; (C) fluorescence image. (B and D) When recCED-3 is expressed using P_{mec-3}cz::ced-3(p17) and P_{mec-18}ced-3(p15)::nz, the touch neurons die and GFP fluorescence is only found in the FLP neurons: (B) differential interference contrast image; (D) fluorescence image.

FIG. 6A-F. Temporal induction of recCaspases using combination of the heat-shock promoter and cell-specific promoters. Expression of just one of the recCaspase-3 subunit form the heat shock promoter (Phsp-16 cz::caspase-3(p17) had no effect on survival of the body wall muscle (A) or touch cells (C), whereas its expression in combination with the other subunit of recCaspase from the muscle-specific promoter [P_{myo-3} caspase-3(p12)::nz] or the touch-cell specific promoter [P_{mec-18} caspase-3(p12)::nz] resulted in apoptosis of the body wall muscles (B) and the touch cells respectively (D). Embryos (C and D) or just-hatched L1 stage larvae (A and B) were heat shocked, and 48 hours later the animals were observed for the death of specific cells. The lower panels in both (A) and (B) show the differential interference contrast photographs, whereas (C) and (D), and the upper panels of (A) and (B) show the fluorescence images; the animals also expressed GFP from P_{myo-3} promoter (A, B) or from P_{mec-18} promoter (C, D). After the heat shock, in animals expressing both the subunits of recCaspase (D), embryonically-derived ALM and PLM touch cells undergo apoptosis, whereas post embryonic AVM and PVM, which are generated in the mid L1 larval stage, are unaffected. (E and F) Induction of cell death at various stages in the life cycle of the animal. Animals were heat shocked as embryos or at various time points after hatching, and 48 hours later were scored for (E) absence of GFP positive touch cells in animals expressing recCaspase-3 from Pₕₛₚ₋₁₆ and P_{mec-18} promoter combination, or (F) for paralysis in the animals that expressed recCaspase-3 from Pₕₛₚ₋₁₆ and P_{myo-3} promoter combination. Data form at least two stable lines and three independent experiments were used to calculate mean and SEM. Control animals expressing single subunit of recCaspase-3 under heat-shock promoter are represented by _ _ _ and -o- lines in (E and F) and _ _ _ line in (F), while the animals expressing both the subunits of recCaspase-3 are represented by solid and _. _ lines in (E and F) and solid line in (F). In (E), death of embryonically derived ALM and PLM touch cells (solid and -o- lines), and post embryonically generated AVM and PVM (_. _ and _ _ _ lines) are plotted separately.

FIG. 7. Time course of induction of recCaspase-3. Animals expressing both subunits of the recCaspase-3 [Pₕₛₚ₋₁₆ cz::caspase-3(p17)+P_{myo-3} caspase-3(p12)::nz] were heat shocked for 2 hours at L1 stage (12 hours after hatching). At the indicated time points after the heat shock, animals were scored as completely paralyzed (blue) or partially paralyzed (red) or as wild type. Data from three stable lines were used to calculate mean and SEM.

FIG. 8. Touch cell death associated with expression of split caspase 9 constructs.

### 5. DETAILED DESCRIPTION OF THE INVENTION

For clarity, and not by way of limitation, the detailed description of the invention is divided into the following sections:

(i) caspases and their subunits;

(ii) binding partners;

(iii) caspase subunit-binding partner constructs;

(iv) uses of caspase subunit-binding partner constructs;

(v) caspase neutralizers; AND

(vi) caspase neutralizers as a “NOT” gate.

### 5.1 Caspases and their Subunits

Caspases in active form, following processing of the procaspase precursor, typically comprise a large (17-20 kDa) and a small (10-12 kDa) subunit. According to the present invention, each subunit, linked to a binding partner, may be expressed separately under the control of different promoters; each component (caspase subunit plus binding partner) is referred to herein as a “Sub-Casp-BP,” and the active product of their assembly is referred to as a “reconstituted caspase” or “recCaspase”. The following describes the structural characteristics of naturally cleaved and assembled caspase subunits, the structure of which may be recapitulated, at least in part, according to the present invention.

When depicted in a linear molecular organization, procaspases contain an N-terminal prodomain followed by a linker to a large subunit precursor followed by another linker region attaching the N-terminal domains to a C-terminally located small subunit precursor. A procaspase is therefore proteolytically processed at a minimum of two aspartic acid maturation cleavage sites to generate three fragments. After cleavage, the catalytic large and small fragments reassemble to form the active enzyme (Funtes-Prior and Salvesen, 2004, Biochem. J. 384:201-232). Structural analysis indicates that the active enzyme is a three-layered twisted 12-stranded β-sheet that is sandwiched by α-helices (i.e. α-helix 1-β-sheet 1 to β-sheet12-α-helix 2). Most of the interdomain contact area is built by the centrally located small subunits so as to form an arrangement of “Large-Small-Small-Large”, with additional interactions tying together the C- and N-termini of large and small subunit domains, to lock the structure into shape. The obligate catalytic domain presents a compact ellipsoid structure composed of a tight alignment of a dimer of two large and two small subunits.

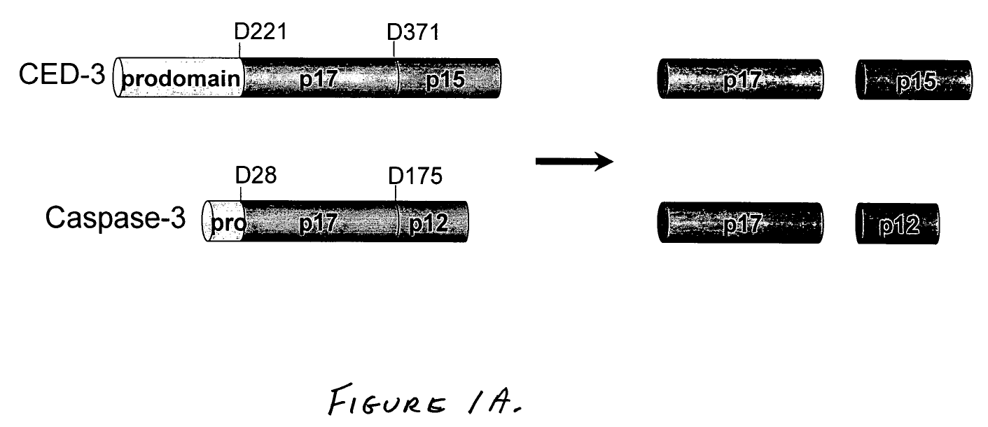

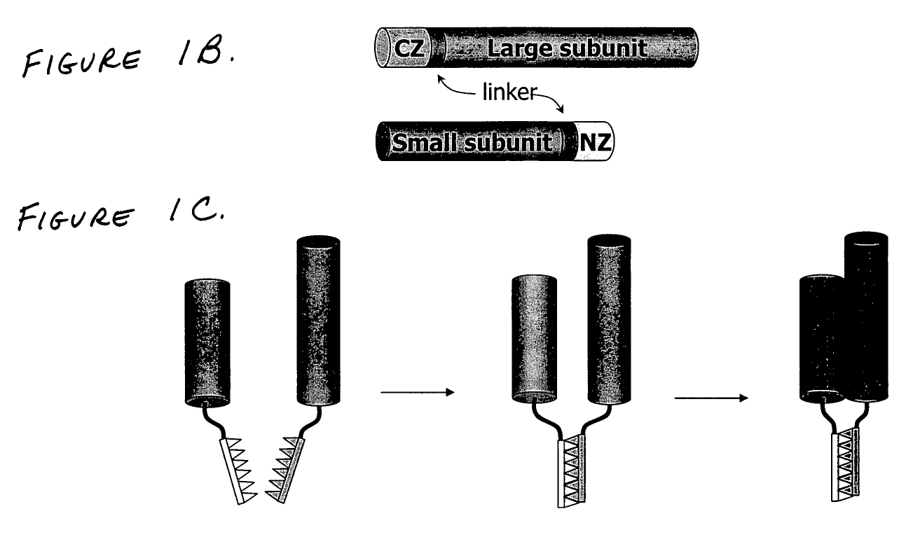

In addition to the constructs exemplified in Example Sections below, and the subunits as set forth for CED-3 and Caspase 3 in FIG. 1, non-limiting examples of caspases which may be used according to the invention, together with the amino acid positions of large and small subunits, are set forth below in Table 1. Preferably, but not by way of limitation, caspases used as a basis for recCaspases according to the invention are executioner caspases.

TABLE 1
Characteristics of mammalian caspases

| | | | | |
|---|---|---|---|---|
| | Size of enzyme | | Active large/small subunit | GenBank |
| | precursor | | size in kDa (amino acid | Accession |
| Enzyme | (kDa) | Prodomain type | coordinates of subunit)^{§} | Number (cDNA) |

Apoptotic initiator caspases

| | | | | |
|---|---|---|---|---|
| Caspase-2 | 51 | Long, with CARD region | 19 (131-297)/12 (311-409) | AY219042 |
| Caspase-8 | 55 | Long, with two DED | 18 (147-297)/11 (313-403) | AB038985 |
| | | regions | | |
| Caspase-9 | 45 | Long, with CARD region | 17 (139-297)/10 (317-403) | AY214268 |
| Caspase-10 | 55 | Long, with two DED | 17 (143-297)/12 (326-409) | BC042844 |
| | | regions | | |
| Caspase-12 | 50 | Long, with CARD region | 20/10 | AF486844 |

Apoptotic effector or executioner caspases

| | | | | |
|---|---|---|---|---|
| Caspase-3 | 32 | Short | 17 (148-297)/12 (315-402) | AY219866 |
| Caspase-6 | 34 | Short | 18 (143-297)/11 (315-405) | AY254046 |
| Caspase-7 | 35 | Short | 20 (119-297)/12 (315-402) | BT006683 |

Inflammatory caspases

| | | | | |
|---|---|---|---|---|
| Caspase-1 | 45 | Long, with CARD region | 20/10 | NM033292 |
| Caspase-4 | 43 | Long, with CARD region | 20/10 | NM001225 |
| Caspase-5 | 48 | Long, with CARD region | 20/10 | NM004347 |
| Caspase- | 42 | Long, with CARD region | 20/10 | BC061255 |
| 11* | | | | |

Other mammalian caspase

| | | | | |
|---|---|---|---|---|
| Caspase-14 | 30 | Short | 0/10 | AF097874 |

*Detected in murine cells.
^{§}Size in kDa and amino acid coordinates of large and small subunits where available.

Caspases appear to follow a hierarchical order of activation starting with extrinsic (originating from extracellular signals) or intrinsic apoptotic signals which trigger the initiator group (caspase-8, 10, 9 or 2) which in turn process the executioner caspases (caspase-7, 3 and 6). While this permits the cell or organism to maintain tight control and regulation of the system, it prevents the ability to experimentally study executioner caspases without triggering upstream processes. The executioner caspases-3 and -6 have been experimentally engineered to generate molecules that are constitutively active in the absence of proteolytic cleavage. These so-called reverse caspases were designed on the basis of the structure of active caspases. Thus it was hypothesized that creation of a single molecule in which the C-terminus of caspase-3 or -6 small subunit when fused to the N-terminus of the corresponding large subunit may generate an active molecule since this arrangement occurs by non-covalent association in native active form of the enzyme. The hypothesis was borne out when engineered reverse caspase-3 (GenBank Accession No. AF052647) and -6 (GenBank Accession No. AF052646) was produced and tested positively in vitro and in vivo (Srinivasula et al., 1998, J. Biol. Chem. 273(17):10107-10111). Reverse caspases-3 and -6 are referred to herein as “REVCasp3” and “REVCasp6”.

### 5.2 Binding Partners

Functionally complementary Sub-Casp-BPs may be assembled to form a recCaspase by a covalent or non-covalent linkage. Complementary binding partners (which can assemble such that two different Sub-Casp-BPs form a recCaspase) may be the same or different. For example, binding partners may be components of a homomeric or heteromeric protein. As another non-limiting example, binding partners may be components of a ligand/receptor pair. Examples of compatible binding partners include, but are not limited to, an antiparallel leucine zipper (as described in United States Patent Application Publication No. 2003/0003506); calmodulin/M13 (as described in Ozawa et al., 2001, Anal. Chem. 73:5866-5874); immunoglobulin (including single chain antibodies and portions thereof)/peptide ligand; hormone/receptor; clathrin, enzyme/substrate; integrins such as alphaIIb and beta3; ubiquitin/ubiquitin interacting motif; viral capsid proteins (e.g., see Barklis et al., 1998, J. Biol. Chem. 273:7177-7120) and other interacting proteins known in the art (e.g., see Xenarius, 2002, Nucl. Acids Res. 30:303-305 regarding the protein interaction database, “DIP” at http://dip.doe-mbi.ucla.edu; Han et al., Bioinformatics, PMID# 15117749 regarding the human protein interaction database http://www.hpid.org; and information available from Biomolecular Interaction Network Database (BIND), Cellzome (Heidelberg, Germany), Dana Farber Cancer Institute (Boston, Mass., USA), the Human Protein Reference Database (HPRD), Hybrigenics (Paris, France), the European Bioinformatics Institute's (EMBL-EBI, Hinxton, UK) IntAct, the Molecular Interactions (MINT, Rome, Italy) database, the Protein-Protein Interaction Database (PPID, Edinburgh, UK) and the Search Tool for the Retrieval of Interacting Genes/Proteins (STRING, EMBL, Heidelberg, Germany). The binding partners may, in the process of association, change structure; for example, the binding partners may comprise an intein together with a member of an interacting pair of proteins (as in Ozawa et al., 2001, Anal. Chem. 73:5866-5874); when the protein pair interact, splicing occurs via the inteins and the interacting pair are cleaved from the now covalently-joined RecCaspase. The binding partners in such embodiments therefore comprises a member of an interacting set of proteins together with an adherent structure that forms a linkage when brought into proximity of a partner structure; in addition to an intein (which produces a covalent linkage), another non-limiting example of an adherent structure (that produces a non-covalent linkage) is a leucine zipper domain.

### 5.3 Caspase Subunit-Binding Partner Constructs

The present invention provides for caspase subunit-binding partner constructs, Sub-Casp-BPs, and for nucleic acid molecules encoding such constructs.

It has been observed that for effective formation of a recCaspase, one binding partner is linked to the small subunit at the N-terminus and the other binding partner is linked to the large subunit at the C terminus (FIG. 1). It is further envisaged that one binding partner may be linked to the small subunit at the C-terminus and the other may be linked to the large subunit at the N-terminus. It may be that other configurations may be possible; for instance, binding partners linked to the N or C termini of both the large and the small subunits may form active recCaspases provided that enough flexibility be present to allow proper assembly of the large and small subunits. For example, a flexible linker peptide sequence may need to be incorporated between the subunit and the binding partner.

The present invention provides for nucleic acids encoding Sub-Casp-BP constructs, operably linked to a promoter of interest. The promoter of interest may be a promoter which is selectively or specifically active in a cell type, including a cell of a particular tissue specificity or at a particular developmental stage, which is to be a target cell according to the invention. A promoter/Sub-Casp-BP expression construct may be assembled in vitro, using standard laboratory techniques. A promoter/Sub-Casp-BP expression construct may be inserted by means known to a skilled artisan such as electroporation, microinjection, ballistic delivery, transfection or transduction, into an animal cell so as to construct a stably expressing cell line of the said construct. In one embodiment, the animal cell is a fertilized oocyte or embryonic stem cell within which the promoter/Sub-Casp-BP expression construct may be inserted at one or more genomic loci to generate a transgenic animal. In an alternative embodiment, a promoterless Sub-Casp-BP may be inserted by “knock-in” technologies (Bremer and Weissleder, 2001, Acad. Radiol. 0.8(1):15-23) into a regulatory region of an endogenous gene within a cell by site specific targeting so that the Sub-Casp-BP is expressed under regulation of the promoter and other regulatory elements of the gene of insertion. In one embodiment, knock-in may result in inactivation of the endogenous gene. Alternatively, utilizing an element such as but not limited to an Internal Ribosome Entry Site (IRES), the endogenous targeted gene as well as a Sub-Casp-BP may be expressed under regulation of the native endogenous promoter. The invention provides for the generation of an animal from the cell in which the promoterless Sub-Casp-BP has been knocked-in by means known to a skilled artisan (Wobus and Boheler, 2005 Physiol. Rev., 85(2):635-78). In yet another embodiment, a promoterless Sub-Casp-BP may be randomly inserted into the genomic DNA of a cell and a transgenic animal generated therefrom to screen or select for a cell specific or tissue specific promoter element based on expression of the Sub-Casp-BP. Thus the invention provides for selective ablation of a specific cell lineage or developmental arrest of an animal due to ectopic expression of a randomly inserted promoterless Sub-Casp-BP construct and the ability to identify a tissue specific or developmental stage specific promoter based on the insertion site of Sub-Casp-BP DNA.

### 5.4 Uses of Caspase Subunit-Binding Partner Constructs

The present invention may be used to demonstrate coordinate activity of promoters that control the expression of complementary Sub-Casp-BP molecules, such that when both promoters are coordinately active so as to produce complementary Sub-Casp-BPs that assemble to form an active recCaspase, apoptosis and/or cell death results. “Coordinate” as used herein means that the promoters are active within a period of time such that their Sub-Casp-BP products co-exist and are capable of assembling to form recCaspase. The use of the term “coordinate” does not require that there be any dependence or direct or indirect functional relationship between the activity of the promoters, although in specific non-limiting examples of the invention, such dependence or relationship may exist. “Coordinate” need not mean “contemporaneous.” However, if promoters driving expression of complementary Sub-Casp-BPs are sequentially active, but the interval between their activity exceeds the life-time of the first Sub-Casp-BP expressed, then their coordinate activity may not be detectable.

Thus, in a host cell containing complementary Sub-Casp-BP expression constructs under the control of different promoters, the promoters may be coordinately expressed if both promoters are active in the host cell type (e.g., tissue specific promoters, constitutively active promoters of “housekeeping” genes) or under conditions to which the host cell is exposed (e.g., changing developmental conditions, changes in extracellular environment, exposure to cytokines, exposure to an inducing agent), including if one promoter is dependent on the gene product of the other for activity.

Thus, in particular, non-limiting embodiments, the present invention provides for a method of detecting coordinate activity of a first and a second promoter element in a host cell containing a first nucleic acid comprising the first promoter operably linked to a nucleic acid encoding a first Sub-Casp-BP and a second nucleic acid comprising the second promoter operably linked to a second nucleic acid encoding a second Sub-Casp-BP, where the first and second Sub-Casp-BPs are complementary, comprising detecting the formation of a recCaspase by detecting indicia of apoptosis (such as, but not limited to, DNA laddering, selective permeability of fluorescent or non-fluorescent dyes e.g. YO-PRO-1, SYTO 13 and SYTO 16, Hoechst 33342, APO-BrdU TUNEL Assay etc.) and/or by detecting cell death (for example, using vital staining, by histologic appearance of dying cells (e.g., a refractile disc-like appearance in C. elegans). The promoters may be different or the same, but preferably the promoters are different.

In particular non-limiting embodiments, the present invention provides for a method of detecting coordinate activity of a first and a second promoter element in a host cell containing a first nucleic acid comprising the first promoter operably linked to a nucleic acid encoding a first split caspase construct comprising a first caspase subunit linked to a first binder element and a second nucleic acid comprising the second promoter operably linked to a second nucleic acid encoding a second split caspase construct comprising a second caspase subunit linked to a second binder element, where the first and second split caspase constructs are complementary, the first and second binder elements can form a bond selected from the group consisting of a non-covalent bond and a covalent bond, and the first and second promoters are not the same, comprising detecting the formation of a reconstituted caspase protein from the split caspase constructs by detecting apoptosis.

In other particular non-limiting embodiments, the present invention provides for a method of selectively inducing apoptosis in a cell type of interest comprising (i) introducing, into a cell of the cell type of interest, a first nucleic acid comprising a first promoter operably linked to a nucleic acid encoding a first split caspase construct comprising a first caspase subunit linked to a first binder element and a second nucleic acid comprising a second promoter operably linked to a second nucleic acid encoding a second split caspase construct comprising a second caspase subunit linked to a second binder element, where the first and second split caspase constructs are complementary, the first and second binder elements can form a bond selected from the group consisting of a non-covalent bond and a covalent bond, and the first and second promoters are selected such that conditions may be provided so that the first and second promoters are selectively active in the cell type of interest, either constitutively or by induction; and (ii) providing conditions such that the first and second promoters are coordinately active such that the first and second split caspase constructs are coordinately expressed and caspase activity and apoptosis in the cell type of interest are induced.

The present invention may be used to selectively ablate cells in which the promoters driving expression of both Sub-Casp-BP constructs are coordinately active. This may be used to select, from a mixed cell population, cells in which both promoters are NOT coordinately active (which would survive). Selective ablation of cells may be performed in a cell culture or in an intact organism (see Example Section 6, below, for experiments performed in intact C. elegans and in HeLa cells in culture). Of note, coordinate promoter activity may not be a natural condition of the cell or organism: for example, and not by limitation, a first promoter driving expression of a Sub-Casp-BP may be inducible (for example, by tetracycline or heat shock) so that ablation of cells in which a second promoter constitutively drives expression of a complementary Sub-Casp-BP, may be induced by adding tetracycline to, or “heat shocking”, the system.

The present invention may be used in therapeutic applications. For example, an expression construct comprising a first promoter, active in a target cell, operably linked to a first Sub-Casp-BP molecule, and an expression construct comprising a second promoter, active in the target cell, operably linked to a second Sub-Casp-BP molecule, which is complementary to the first Sub-Casp-BP molecule, may be introduced into the target cell, optionally contained in the same vector molecule (e.g., an adenoviral vector). The promoters may be constitutively active in the target cell (for example, where the target cell is a cancer cell, and both promoters are selectively or specifically active in cancer cells) or one or both promoters may be inducibly active.

In additional non-limiting embodiments, the present invention may be used to produce animal models of human diseases. The present invention provides for a non-human animal model of disease associated with depletion or dysfunction of a target cell comprising an animal containing a first transgene comprising a first Sub-Casp-BP operably linked to a first promoter active in the target cell and a second Sub-Casp-BP operably linked to a second promoter active in the target cell, wherein the first and second Sub-Casp-BPs are complementary and selectively lead to the death of target cells in the animal. For example, but not by way of limitation, recCaspase may be used to selectively ablate pancreatic islet cells in a mouse (using islet cell specific promoters to drive expression of complementary Sub-Casp-BPs) or renal podocytes (using podocyte-specific promoters to drive expression of complementary Sub-Casp-BPs) to provide murine models of diabetes and renal disease, respectively.

If, as set forth in section 10 below, complementary Sub-Casp-BPs are used to reconstitute activity of caspase 9, it may be desireable to provide, in the cell in which the Sub-Casp-BPs are expressed, procaspase 3.

### 5.5 Caspase Neutralizers

The present invention further provides for the use of CAspase NeuTralizer (“CANT”) molecules, the expression of which may be controlled by a promoter of interest. CANT molecules that may be used according to the invention include, but are not limited to, baculovirus p35 protein, baculovirus p49 protein, CrmA, members of the Inhibitors of Apoptosis family, vFLIP proteins as encoded by herpesvirus or molluscpox virus, and RNAi directed at a Sub-Casp-BP or REV-Casp (Funtes-Prior and Salvesen, 2004, Biochem J. 384:201-232; Bode and Huber, 2000, Biochim. Biophys. Acta. 1477:241-252; Thome et al., 1997, Nature 386:517-521; Hu et al., 1997, J. Biol. Chem. 272:9621-9624; Gettins, 2002, Chem. Rev. 102:4751-4804; Jabbour et al., 2002, Cell Death Differ. 9:1311-1320; Zoog et al., 2002, EMBO J. 21:5130-5140; Bode and Huber, 2000, Biochim. Biophys. Acta. 1477:241-252; Crook et al., 1993, J. Virol. 67:2168-2174; Deveraux and Reed, 1999, Genes Dev. 13:239-252; Kreuter et al., 2004, Arch. Immunol. Ther. Exp., 52:141-155; Kawasaki et al., 2000, Am. J. Pathol. 157:597-603; Zender et al., 2003, Proc. Natl. Acad. Sci. U.S.A., 98:7797-7802).

### 5.6 Caspase Neutralizers as “Not” Gates

The present invention further provides for detecting coordinate activity of more than two promoters. For example, the method set forth above may be altered so that in addition to two promoters controlling the expression of complementary Sub-Casp-BPs, there is a third promoter controlling the expression of a Caspase Neutralizer (“CANT”), as set forth above. While cells expressing only complementary Sub-Casp-BPs will form active caspase and die, cells in which all three promoters are coordinately active will also express CANT and, provided that sufficient CANT is available, will not apoptose and die.

In related embodiments of the invention, coordinate activities of (i) a promoter controlling expression of a REV-Casp molecule such as, but not limited to, REV-Casp-3 or REV-Casp-6 and (ii) a promoter controlling expression of a CANT molecule may be evaluated, wherein expression of the first promoter only (driving REV-Casp expression) may result in cell apoptosis and/or death, but expression of both promoters, where sufficient CANT is produced, may rescue the cells from apoptosis and/or death.

In certain non-limiting embodiments of the invention, at least one of the promoters controlling expression of Sub-Casp-BPs or CANT is conditionally expressed (for example, inducible).

### 6. EXAMPLE

### Oligomerization of Individually-Expressed Caspase Subunits is Needed for Cell Killing

### 6.1 Materials and Methods

Nematode protocols: Animals were maintained, until otherwise mentioned, at 20° C. as described (Brenner, 1974). Transgenic animals were generated by microinjection into wild type (N2), TU2769 (uIs31), TU2770 (uIs32) [these strains contain different integrated insertions of mec-17::gfp, which expresses GFP specifically in the touch neurons (O'Hagan et al., 2005)], or TU2973 [ced-4(n1162), uIs32]. The expression plasmids (50 μg/ml if injected alone or 25 μg/ml if two were injected) were injected with the dominant roller plasmid, pRF4 (50 μg/ml) that serves as the transformation marker (Mello et al., 1991). At least three stable lines were obtained for each genotype. The extrachromosomal array was integrated into the chromosome following the slightly modified integration protocol of I. Greenwald and O. Hobert (personal communication). Animals were irradiated with gamma rays (4800 rads) and lines that inherited the transformation marker 100% of the times in the subsequent generations were selected.

Expression Constructs: The sequences corresponding to the anti-parallel leucine zipper domains NZ (ALKKELQANKKELAQLKWELQALKKELAQ) (SEQ ID NO:1) and CZ (AQLEKKLQALEKKLAQLEWKNQALEKKLAQ) (SEQ ID NO:2) along with the linker sequence “GGSG” were amplified from bacterial expression plasmids pET11a-NZGFP and pET11a-CZGFP(Ghosh et al., 2000) (a gift from Lynne Regan). The sequences corresponding to p17 and p15 subunits of CED-3 (FIG. 1A) were amplified either from genomic DNA or from P_{mec-7}acCED-3 (a gift from Ding Xue,) which contains a mutation that results in a constitutive ced-3 activity(Parrish et al., 2001). The sequences for p17 and p12 subunits of Caspase-3 (FIG. 1A) were amplified from a human Caspase-3 cDNA (Mammalian Gene Collection full length cDNA clone ID 4419175). All the constructs used for C. elegans expression were derived from the promoter-less GFP plasmid pPD95.75 (a gift from Andy Fire; www.ciwemb.edu/pages/firelab.html). Plasmid constructs used for expression in HeLa Tet-ON cell line were derived from pTRE-Tight (Clontech), which contains a tetracycline-responsive promoter. Details about the cloning of expression constructs are provided in the Supplemental material.

Cell death assay: In C. elegans the death of the touch receptor neurons, which were labeled with GFP, was monitored by the loss of GFP fluorescence in adult worms under a Leica stereo dissection microscope equipped for fluorescence microscopy. L1 larvae (collected 2-4 hrs after hatching) were observed using a Zeiss Axioscope 2 microscope. The percent of surviving cells were calculated by dividing the number of GFP positive cells by the total number of touch cells (number of animals X 6 for adults and X 4 for the L1 larvae). To confirm the Ced phenotype, late embryos (3-fold stage) or early L1 larvae were observed under Nomarski differential interference contrast optics for the presence cell corpses with the flat, refractile disc-like appearance that is characteristic of apoptosis in C. elegans (Sulston and Horvitz, 1977). To examine the effect of ced mutations on recCaspase activity, we generated lines for ced-3(n717), ced-4(n1162) and ced-8(n1891) that contained integrated copies of recced-3 or recCaspase-3 and mec-17::gfp. The Ced phenotype of these animals was confirmed by the absence of all non-touch neuron cell corpses in the embryos for ced-3 and ced-4 genes (Ellis and Horvitz, 1986), and by the absence of cell corpses in the bean and comma stage of early embryos and the presence of more than ten corpses in the head of 3-fold embryos for ced-8 (Stanfield and Horvitz, 2000).

Caspase activity in HeLa cells: HeLa Tet-On cells were maintained in DMEM medium with 10% Tet system approved FBS (BD Biosciences). Cells cultured in six-well plates were transiently transfected using Lipofectamine 2000 (Invitrogen). The DNA mix contained plasmids for Caspase-3 subunits with or without leucine zippers or the control vector plasmid (pTRE-Tight) along with the pCaspase3 sensor EYFP vector (Clontech) and the plasmid encoding the tetracycline transactivator rtTA2-M2 (Urlinger et al., 2000). Cells were split eight hours after transfection, plated on coverslips and allowed to grow for another 12 hours, at which time doxycycline (11 g/ml) was added to start the induction of expression. Cells were fixed at 12 hours after induction and the percentage of cells with caspase activity was determined by the number of cells with nuclear localized GFP divided by the total number of fluorescent cells. More than 300 green cells were counted from randomly chosen fields in each experiment; data from three independent experiments were used to calculate mean and standard deviation. EGFP expressed from TRE-Tight promoter showed a highly inducible doxycycline-dependent expression under similar experimental condition.

TABLE S1
Supplementary material:
Description of the plasmids and their construction

| Plasmid | Description of the plasmid | Insert* | Vector | Primers |
|---|---|---|---|---|
| TU# 739 | P_{mec-18}yfp | 0.4 kb HindIII-BamHI PCR fragment | pPD95.75yfp^{#} | P1 + P2 |
| | | encoding the mec-18 promoter | | |
| TU# 798 | Pₙₘᵣ₋₁yfp | 1.2 kb XhoI-BamHI PCR fragment | TU# 739 | P3 + P4 |
| | | encoding the nmr-1 promoter | | |
| TU# 799 | P_{cfi-1}yfp | 5.4 kb Xhol-BamHI PCR fragment | TU# 739 | P5 + P6 |
| | | encoding the cfi-1 promoter | | |
| TU# 800 | P_{mec-18}ced-3(p15,)^{†} | BamHI-EcoRI PCR fragment encoding | TU# 739 | P7 + P8 |
| | | the ced-3(p15) | | |
| TU# 801 | P_{mec-18}ced-3(p17)^{†} | BamHI-EcoRI PCR fragment encoding | TU# 739 | P9 + P10 |
| | | the ced-3(p17) | | |
| TU# 802 | P_{mec-18}cz::yfp | BamHI-Xmal PCR fragment encoding | TU# 739 | P11 + P12 |
| | | the cz sequence | | |
| TU# 803 | P_{mec-18}nz | KpnI-EcoRI PCR fragment encoding | TU# 739 | P13 + P14 |
| | | the nz sequence | | |
| TU# 804 | P_{mec-18}ced-3(p17)::nz^{†} | BamHI-XmaI PCR fragment encoding | TU# 803 | P9 + P16 |
| | | ced-3(p17) | | |
| TU# 805 | P_{mec-18}cz::ced-3(p15^{†} | KpnI-EcoRI PCR fragment encoding | TU# 802 | P8 + P15 |
| | | ced-3(p15) | | |
| TU# 806 | P_{mec-18}ced-3(p15)::nz | BamHI-XmaI PCR fragment encoding | TU# 803 | P7 + P17 |
| | | ced-3(p15) | | |
| TU# 807 | P_{mec-18}cz::ced-3(p17)^{†} | AgeI-EcoRI PCR fragment encoding | TU# 802 | P10 + P18 |
| | | ced-3(p17) | | |
| TU# 808 | P_{mec-18}ced-3(p15)::nz | BamHI-KpnI PCR fragment encoding | TU# 806 | P7 + P19 |
| | | wt-ced-3(p15) | | |
| TU# 809 | P_{mec-18}cz::ced-3(p17) | SmaI-EcoRI PCR fragment encoding | TU# 807 | P10 + P20 |
| | | wt-ced-3(p17) | | |
| TU# 810 | P_{cfi-1}ced-3(p15)::nz^{†} | PvuI + Bam digested fragment of | TU# 806 | |
| | | TU#799 containing cfi-1 promoter | | |
| TU# 811 | Pₙₘᵣ₋₁cz::ced-3(p17)^{†} | HindIII + Bam digested fragment of | TU# 807 | |
| | | TU#798 containing nmr-1 promoter | | |
| TU# 812 | P_{mec-3}cz::ced-3(p17)^{†} | BamHI-XhoI PCR fragment containing | TU# 807 | P21 + P22 |
| | | mec-3 promoter | | |
| TU# 813 | P_{mec-18}Caspase-3(p12)::nz | BamHI-XmaI PCR fragment encoding | TU# 806 | P23 + P24 |
| | | Caspase-3(p12) subunit | | |
| TU# 814 | P_{mec-18}cz::Caspase-3(p17) | Kpn1-EcoRI PCR fragment encoding | TU# 807 | P25 + P26 |
| | | Caspase-3 (p17) | | |
| TU# 815 | P_{TRE-Tight}Caspase-3(p12) | BamHI-XbaI PCR fragment encoding | pTRE-Tight | P24 + P31 |
| | | Caspase-3(p12) subunit | | |
| TU# 816 | P_{TRE-Tight}Caspase-3(p17) | BamHI-XbaI PCR fragment encoding | pTRE-Tight | P26 + P32 |
| | | Caspase-3(p17) subunit | | |
| TU# 817 | P_{TRE-Tight}Caspase-3(p12)::nz | BamHI+MscI digested PCR fragments + MscI + XbaI | BamHI + XbaI | P23 + P24 |
| | | digested PCR fragment. | digested pTRE- | P27 + P28 |
| | | | Tight | |
| TU# 818 | P_{TRE-Tight}cz::Caspase-3(p17) | BamHI + KpnI digested PCR fragment + KpnI + XbaI | BamHI + XbaI | P29 + p30 |
| | | digested PCR fragment | digested pTRE- | P25 + P26 |
| | | | Tight | |

*The templates used for amplification of inserts were: NZ and CZ from pET11a-NZGFP and pET11a-CZGFP (Ghosh et al., 2000), p17 and p15 subunits of CED-3 from genomic DNA or from P_{mec-7}acCED-3 (Parrish et al., 2001), p17 and p12 subunits of Caspase-3 from a human Caspase-3 cDNA (MGC clone ID 4419175), the promoter sequence of mec-3 from the pPD57.56 (Andy Fire vector), and other promoter sequences from genomic DNA.
^{#}All the constructs used for C. elegans expression were derived from pPD95.75 in which the gfp sequences were replaced by the sequence of yfp from pPD133.58.
^{†}These constructs were derived from acCED-3. We have not found any functional difference between these and the corresponding wild-type subunits.

TABLE S2
primer sequence information

| | | |
|---|---|---|
| Primer | Primer sequence (5′→3′) | SEQ ID NO: |

| | | |
|---|---|---|
| P1 | TCCGAAGCTTCAATTAATTCGTCTACTATCC | 3 |
| P2 | TTATGGATCCGCTCACAACCTTCTTGGAAG | 4 |
| P3 | TTATACTCGAGAAAATGCGTTCCCACTTCTTG | 5 |
| P4 | ATATAAGGATCCATCTGTAACAAAACTAAAGTTTGTCGTG | 6 |
| P5 | GTATACTCGAGGATGATGATTGAAATTTGAGAACGA | 7 |
| P6 | GATGTGGATCCTGCAAGAAAATACAAACTCTTAGAATTCA | 8 |
| P7 | ATCAGGATCCAAAATGGGAGTTCCTGCATTTCTTC | 9 |
| P8 | GAATCACGAGTGAATTCTAGACGGCAGAG | 10 |
| P9 | TATCAGGATCCAAAATGGCACCAACCATAAGCCGT | 11 |
| P10 | AGTTAGAATTCTCAGTCGACAGAATCCAAGAC | 12 |
| P11 | TATCAGGATCCAAAAATGGCTAGCGCACAGCTG | 13 |
| P12 | TTATTACCCGGGGACCGCTTCCACCCTGTGC | 14 |
| P13 | TTATTAGGTACCAGGCTCTGGCTCTGGCGC | 15 |
| P14 | TCAGTTGGAATTCTCACTGAGCCAGT | 16 |
| P15 | TTATTAGGTACCAGGAGTTCCTGCATTTCTTC | 17 |
| P16 | TTATTACCCGGGGGTCGACAGAATCCAAGAC | 18 |
| P17 | TTATTACCCGGGGGACGGCAGAGTTTCGTGCTT | 19 |
| P18 | TTATTAGGTACCGGTAGCACCAACCATAAGCCGTG | 20 |
| P19 | TTATTAGGTACCGGGACGGCAGAGTTTCGTGCTT | 21 |
| P20 | TATCACCCGGGCGAAGATGGCACCAACCATAAGCCGTG | 22 |
| P21 | TTACTGGATCCGTAGTTCAAATGAAATAAATCAGAAG | 23 |
| P22 | TGGATCTCGAGTAGTTGGCGAAGATAGAAATGG | 24 |
| P23 | TAATACCCGGGGGTGATAAAAATAGAGTTCTTTTGTGAGC | 25 |
| P24 | TATCAGGATCCGCCACCATGAGTGGTGTTGATGATGACATGG | 26 |
| P25 | TTATTAGGTACCAATGGAGAACACTGAAAACTCAGTGG | 27 |
| P26 | CTCCTGAATTCTAGATCAGTCTGTCTCAATGCCACAGTCC | 28 |
| P27 | ACATAATGGCCAAAGGAGGACCCTTGGAGGGTACC | 29 |
| P28 | TCCACTCTAGATCACTGAGCCAGTTCTTTCTTCAGTGC | 30 |
| P29 | TATTAGGATCCGCCACCATGGCTAGCGCACAGCTGGAGAAGAAACTGC | 31 |
| P30 | ATAATAGGTACCTCCTTTGGGTCCTTTGGCCAATCC | 32 |
| P31 | TATAGTCTAGATCAGTGATAAAAATAGAGTTCTTTTGTGAGC | 33 |
| P32 | TATCAGGATCCGCCACCATGGAGAACACTGAAAACTCAGTGG | 34 |

### 6.2 Results and Discussion

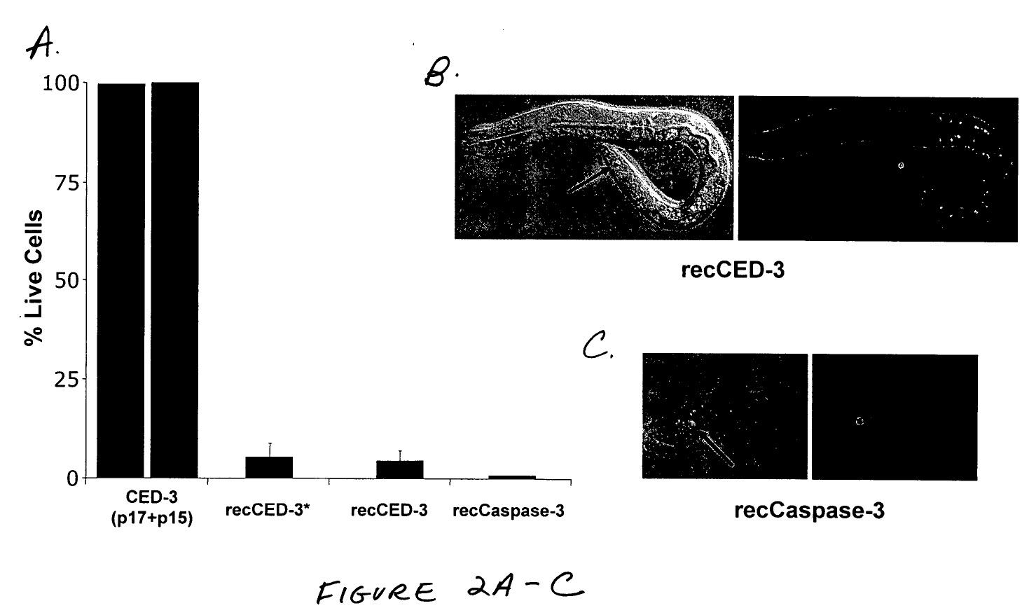

Active caspases are generated from procaspases by cleavage at conserved aspartate residues in the linker region connecting the two subunits ((Cohen, 1997), FIG. 1a). The C. elegans ced-3 gene encodes a caspase needed for programmed cell death (Ellis and Horvitz, 1986; Xue et al., 1996). To test if we could reconstitute CED-3 caspase activity by expressing the individual subunits in the same cell, we expressed the small and the large subunits of CED-3 ((Xue et al., 1996) separately under the control of mec-18 promoter. This promoter is expressed only in six touch receptor neurons of C. elegans (G. Gu and M. Chalfie, unpub. data). The DNAs for both subunits were injected into a strain whose touch receptor neurons were labeled with GFP, so that the loss of the cells could be monitored by the loss of GFP fluorescence. No loss of GFP fluorescence was detected in injected strains (3 stable lines; 25 animals/line) suggesting that the expression of the caspase subunits by themselves did not result in significant death of the touch sensory neurons (FIG. 2a). Because C. elegans transformation usually results in the production of extrachromosomal arrays that can be lost during cell division or production of germ cells, we created two lines in which the transformed DNA integrated into one of the C. elegans chromosome and, thus, was found in every cell. No touch receptor loss was detected in either line.

This failure to cause cell death may be due to their inability either to associate or to fold properly into their final conformation. To distinguish between these possibilities and promote the association of the subunits, we included interacting anti-parallel leucine-zipper domains to each subunit. These domains can reconstitute fluorescent GFP from split polypeptides (Ghosh et al., 2000; Zhang et al., 2004). We added the leucine zipper domains to the N-terminus of the large and C-terminus of the small subunits (FIGS. 1b,c) based on the x-ray crystallographic structures of Caspase-3 and Caspase-7 (Chai et al., 2001; Mittl et al., 1997; Rotonda et al., 1996) and the production of activated “reverse” Caspase-3 and Caspase-6 by Srinivasula et al., 1998. These studies indicate that the N-terminus of the large subunit and the C-terminus of the small subunits are close together. In contrast to the result of the unmodified subunits, expression of the leucine-zipper-caspase subunits from the mec-18 promoter caused the death of more than 60% of the touch sensory neurons as monitored by the loss of GFP fluorescence (FIG. 2a; 4 stable lines; 20 animals/line). Since the stability of extrachromosomal array formed by the injected DNA is quite variable, we generated lines in which the injected DNA is stably integrated into the chromosomal DNA. In such integrated lines, death of the touch sensory-neuron was more than 95% (FIG. 2a). This loss of GFP expression was due to apoptotic death of the touch neurons, since cell corpses with the distinct flat, refractile disc-like appearance that is characteristic of apoptosis in C. elegans (Sulston and Horvitz, 1977), were clearly visible in the positions of touch neuron cell bodies in late embryos or early L1 larvae (FIG. 2b); many of these apoptotic cells, at this stage, retained a low level of GFP fluorescence. Transformation of either leucine-zipper domain-caspase subunit by itself did not result in touch receptor loss (data not shown). The position of the leucine zipper domains was important, since no cell loss was seen when they were placed in the opposite orientation, i.e., at the end of C-terminus of large subunit and N-terminus of the small subunit (96±4% GFP positive cells; 4 stable lines; 20 animals/line). Our results suggest that the subunits in CED-3 are similarly oriented as they are in other caspases.

A similar activated form of the human Caspase-3 enzyme could also be generated. Caspase-3 belongs to the class of executioner/effector caspases that remain inactive until cleaved by an upstream initiator caspase. Since C. elegans does not have a caspase cascade and lacks initiator caspases, we first tested if Caspase-3 activity could be reconstituted by expressing leucine-zipper-caspase subunits in C. elegans from the mec-18 promoter. Expression of the recombinant human caspase-3 subunits caused apoptotic death of the touch sensory neurons in about 80% of the cells (FIG. 2a; 3 lines; 30 animals/line). Chromosomal integration of the transformed DNA resulted in death of nearly 100% of the touch receptor neurons (FIG. 2a). Caspase-3-mediated and ced-3-mediated touch cell deaths were indistinguishable from each other both in terms of the morphology (FIG. 2c) of the dying cells as well as in timing of the cell death. Since human Caspase-3 was at least as efficient as CED-3 in causing cell death in C. elegans, it is likely to act on the same set of downstream targets.

To determine if the induced cell death was entirely due to the constitutive activity of the reconstituted caspases (recCaspase) or if it required known endogenous components of the cell death pathway, we transferred the integrated arrays expressing either recCED-3 or recCaspase-3 into ced-3(n717), ced-4(n1162) and ced-8(n1891) mutants. ced-4, an ortholog of the mammalian caspase activator Apaf-1, acts upstream of ced-3 and functions as the activator of ced-3. Loss of ced-4 activity (as of ced-3) prevents programmed cell death in C. elegans (Ellis and Horvitz, 1986). In contrast the ced-8 gene is postulated to act downstream of ced-3 to increase the efficacy of cell killing (Stanfield and Horvitz, 2000). Loss of ced-8 activity delayed appearance of cell corpses during embryonic development.

Loss of endogenous ced-3 did not reduce the recCED-3 or recCaspase-3 mediated death of the touch receptor neurons indicating that it is not required for recCaspase activation (Table 2). (Presumably the recCaspases are capable of activating the endogenous CED-3 in wild-type animals.). Similarly, loss of endogenous ced-4 did not affect recCapase-3 killing (Table 2). The integrated recCED-3 array and ced-4 mapped to the same chromosome, making the construction of the combined strain difficult. Instead we looked for effects of extrachromosomal recCED-3 arrays transformed into ced-4 mutants; similar amounts of cell death were seen in two different lines (data not shown). These observations are consistent with the model of induced proximity for ced-3 activation (Salvesen and Dixit, 1999; Yang et al., 1998). According to the model, the binding of ced-4 to ced-3 and subsequent oligomerization of ced-4 brings the ced-3 molecules to close proximity, which facilitates autoproteolytic activation. Since the recombinant molecules do not require proteolytic activation, absence of ced-4 would have no effect on the activity of such molecules.

Although CED-4 is not needed for recCaspases activity, we tested whether it might be needed for the activity of the caspase subunits without the leucine zipper domains. Our results indicate that separate expression of caspase subunits can produce a active enzyme if they can be brought together in the correct orientation. A similar conclusion can be drawn from the “reverse” human and Drosophila caspases, which are constitutively active when the N-terminus of the large subunit is covalently linked to the C-terminus of the small subunit (Srinivasula et al., 1998; Wang et al., 1999) The need for association may be provided by the caspase zymogen or may reflect the involvement of another associated protein (Chinnaiyan, 1999). CED-4 is a appealing candidate for this function, since it and its mammalian ortholog Apaf1 have been implicated in cell-death caspase activation (Chinnaiyan et al., 1997), it binds to the prodomain and protease domains of proCED-3 (Chaudhary et al., 1998), and studies of non-dividing mammalian cells have identified a requirement for increased Apaf1 expression for cytochrome C-induced apoptosis (Wright et al., 2004). Transformation of wild-type CED-4, however, did not increase the number of cells deaths when added to the lines expressing caspase subunits (without the leucine-zipper domains) were expressed from integrated chromosomal sites (6 stable lines).

The number of surviving touch receptor neurons in adults of ced-8 mutants expressing recCED-3 was not different from the wild-type controls (Table 2). In contrast, newly hatched ced-8 larva had many more GFP-positive cells than wild-type or ced-3 animals. These results are consistent with those of Stanfield et al. (Stanfield and Horvitz, 2000), indicating that mutation of ced-8 delays the onset of programmed cell death. Since the recCED-3 is constitutively active, our results suggest that ced-8 acts downstream of ced-3 to increase the efficiency of cell death and it is not required for activation of CED-3.

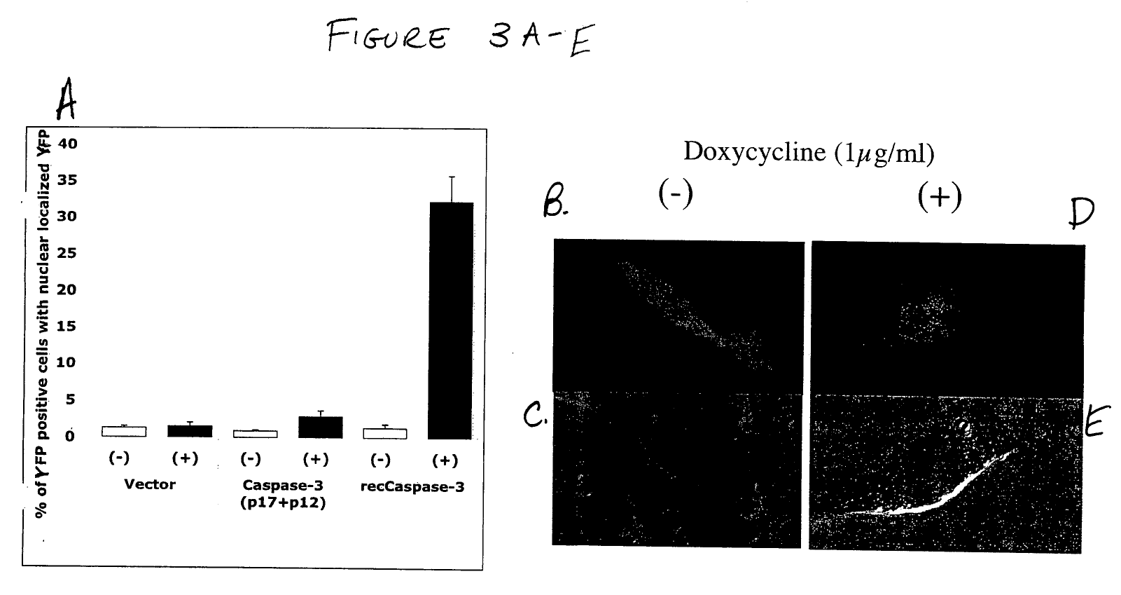

RecCaspase activity is not restricted to C. elegans as seen by the inducibility and in vivo activity of recCaspase-3 under the tightly regulated Tet-inducible promoter in transiently-transfected HeLa cells. To monitor caspase activity, we also cotransfected a caspase-sensing EYFP vector. The resulting YFP has a Caspase-3-specific cleavage site between YFP and a nuclear export signal and a nuclear localization signal at the C-terminus of YFP. The nuclear export signal is dominant over the nuclear localization signal, keeping YFP out of the nucleus. Cleavage by Caspase-3 removes the nuclear export signal, allowing YFP to go to the nucleus. In the uninduced cells, less than 2% of the YFP positive cells had protein localized to the nucleus (FIG. 3a). Induction of recCaspase expression with doxycycline increased this number to 30-40% within 8-12 hours (FIGS. 3a,b). Induction of Caspase-3 subunits without the leucine zipper domains did not show any significant increase in apoptotic activity (FIG. 3a).

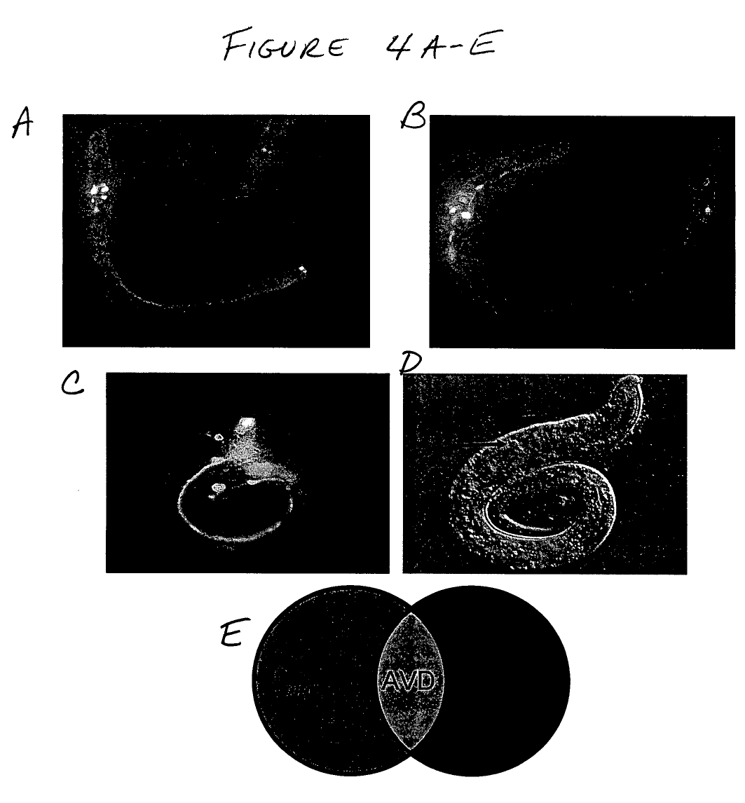

Because the recCaspases are two component systems that lead to cell death, the two parts can be expressed from different promoters to selectively ablate only that subset of cells that expresses both promoters. Our lab has previously described the use of the two-component recGFP system to selectively label subsets of cells (Zhang et al., 2004). To demonstrate the usefulness of this approach, we have constructed animals in which only the AVD interneurons die in the head of C. elegans. The touch receptor neurons in the anterior of the animal form gap junctions onto the AVD interneurons and chemical synapses onto the AVB interneuron (Chalfie et al., 1985). These interneuronal connections are redundant (Chalfie et al., 1985), so mutant affecting one set of connections cannot be easily identified by the loss of touch sensitivity. Unfortunately, no gene is yet known to be expressed only in the AVD cells in the head, so that only these cells could be eliminated. We were able, however, to generate animals lacking the AVD cells by expressing the two parts of recCED-3 from the nmr-1 promoter, which is expressed in AVA, AVD, AVE, RIM, AVG, PVC neurons (Brockie et al., 2001), and from cfi-1 promoter, which is expressed from IL2, URAD, URAV, AVD and PVC, LUA neurons (Shaham and Bargmann, 2002) (FIG. 4a-e). By stably integrating the injected DNA into the chromosome, we will able to establish a strain of C. elegans that will be genetically ablated for AVD. Such a strain will be useful not only for further genetic analysis, but also for analysis of neuronal circuits. We also envision using the recCaspases with GFP and recGFP expressed from other promoters to further restrict fluorescent protein expression in multiple component systems.

These systems can also be used in other organisms. Using a combination of promoters, one of which is expressed in specific cells or tissue and the other whose expression can be regulated by an inducer, not only specific cells can be targeted for killing but also timing of induction of cell death can be regulated. Alternatively using two promoters that target the same cells or tissues could insure tight regulation of caspase activity or cell death.

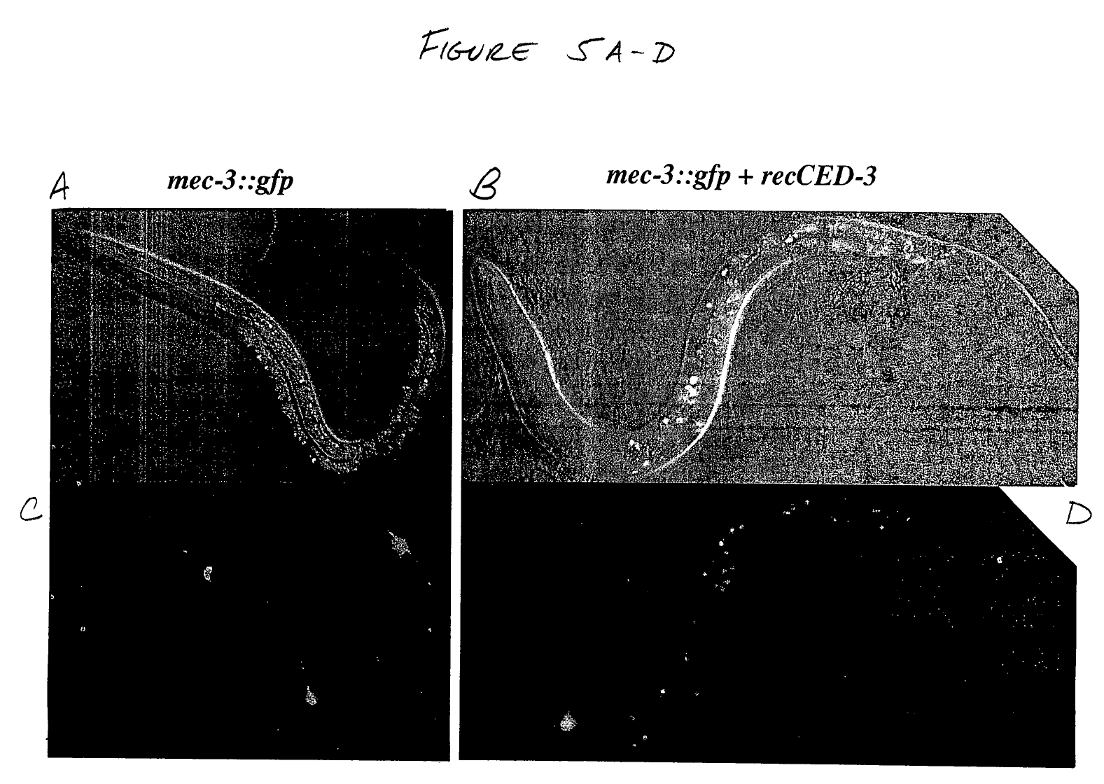

recCaspase killing can also be used to eliminate unwanted cells from a set of labeled cells. For example, no promoter has been identified in C. elegans that uniquely labels the two FLP neurons. A mec-3::gfp fusion, however, is expressed in the FLP neurons and touch neurons in embryos and newly hatched animals (Way and Chalfie, 1989). By expressing the two subunits of recCED-3 from the mec-3 and mec-18 promoters, we were able to kill the touch receptor neurons, which express both promoters, but not the FLP neurons in animals expressing the mec-3::gfp fusion (FIG. 5a-d). Only FLP cells are tagged with GFP in embryos and early larval stages of the resulting animals.

### 8. EXAMPLE

### Applicability to C. elegans

The C. elegans promoter expression database (http://wormbase.org/db/searches/expr_search) and (http://chinook.uoregon.edu/promoters.html) was surveyed to evaluate approximately how many cell types could be selectively marked using a single promoter or a combination of promoters. A C. elegans hermaphrodite has 302 neurons which can be grouped into 113 groups (White et al., 1986). Only 12% of these groups can be marked by cell-specific promoters (Table 3) An additional 70% of the neuron groups can be marked by using combination of two promoters and thus, can be specifically killed by using the dual component recCaspases. At present, only 18% of the C. elegans neurons cannot be selectively killed by this method using the available promoters.

### 9. EXAMPLE

### Temporal Induction OF recCASPASES

### 9.1 Materials and Methods

Experiments were carried out essentially as above, except that for heat shock constructs, the expression plasmids were injected at a reduced concentration of 10 μg/ml (for each construct) along with 80 μg/ml of roller plasmid. At least three stable lines were obtained for each genotype.

The death of body wall muscle cells, which resulted in the paralysis of the animals, was scored as percentage of animals that were paralyzed two days after heat shock. The change in the morphology of the dying body wall muscle cells, which were also labeled by Pmyo-3::gfp in transgenic animals, was observed using a Zeiss Axioscope 2 microscope.

Heat shock experiments: The animals used for heat shock experiments were grown at least for two generations at 15° C. prior to heat shock to minimize the back ground level of expression from the heat shock promoter, which are present in multiple copies in the injected DNA array. At the specified stages, the animals were heat shocked by incubating them at 34° C. for two hours and immediately after the heat shock, the animals were transferred back to 15° C. Unless indicated otherwise, the death of touch cell or the muscle cells were scored 48 hours after heat shock.

### 9.2 Results

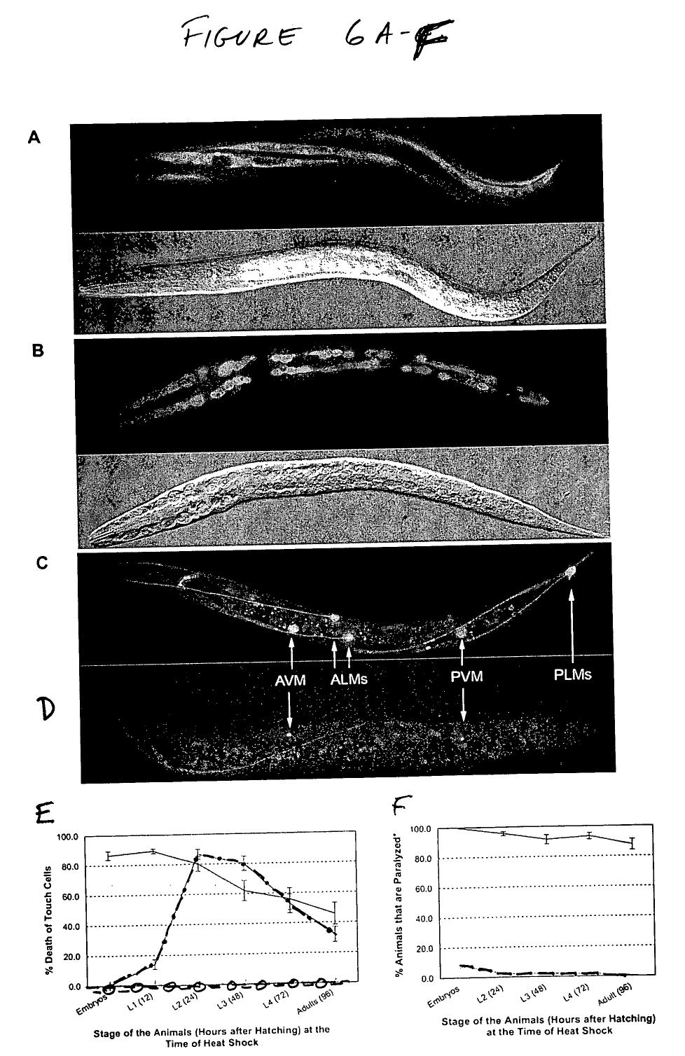

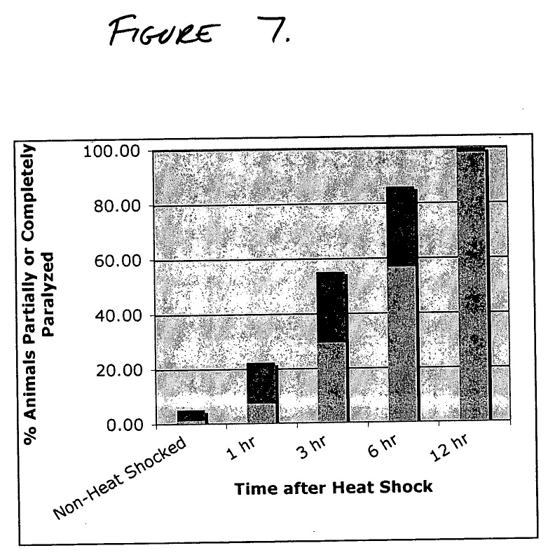

The dual component nature of the recCaspase was exploited to induce cell death in specific cells at specific developmental stages of the animal's life cycle. The inducible heat shock promoter (hsp-16) was used in combination with either the touch cell specific promoter (mec-18) or the body wall muscle cell specific promoter (myo-3) to express recCaspase-3 at specific time points. The cell specific promoter is expressed constitutively throughout development but only in specific cells, while the heat shock promoter is widely expressed but only for a short time after heat shock. Heat shocking the animals that expressed recCaspase-3 from the combination of hsp-16 and myo-3 promoters, paralyzed more than 90% of animals (FIG. 6F). There was only a very small drop in efficiency of recCaspase induction in adult worm compared to the L1 larval stage, which reflects the strong and consistent expression of myo-3 promoter throughout development. Upon heat shock the large muscle cells rounded up, accumulated large number of vacuoles, and the nuclei showed flattened disc-like refractile appearance that is characteristic the apoptotic cells (FIG. 6B). Less than 1% of the non heat-shocked animals grown at 15° C. showed any kind of movement defect or paralysis. This indicates that the hsp-16 promoter is very tightly regulated, and the expression of the small subunit (p12) of recCaspase-3 by itself, even from a very strong promoter such as myo-3, is non toxic to the cells. Similarly the expression of the large subunit (p17) of recCaspase-3 alone from hsp-16 promoter upon heat shock did not cause any paralysis of the animal or changes in the morphology of the muscle cells (FIGS. 6A and 6F). Similar results were obtained from experiments using animals expressing heat shock inducible recCaspase-3 in touch cells. Although death could be specifically induced in touch-cell at all stage of development, there was a marked drop in the efficiency of induced cell death in adult animals compared to L1 larvae (FIG. 6E). These results are consistent with the reduced activity of the mec-18 promoter in adult animals compared to early larval stages (Ma and Chalfie; unpublished results). These observations point out the importance of promoter strength and its temporal expression pattern in the regulation of recCaspase activity. Induction of recCaspase-3 expression in the embryo, lead to very efficient killing of only embryonically derived ALM and PLM touch neurons (>90%), while virtually none of the AVM and PVM (generated postembryonically in mid L1 larval stage) were affected (FIG. 6D). Our results clearly indicate that recCaspase activity can be very tightly regulated and induced in a stage specific manner.

To find out how long it takes for the recCaspase activity to be induced, the activity of heat shock induced recCaspase-3 (expressed from the combination of myo-3 and hsp-16 promoters) was evaluated in muscle cells. L1 larvae (12 hours after hatching at 15° C.) were heat shocked for two hours and the number of animals that were either completely or partially paralyzed were counted. Just within three hours after induction of heat shock, more than 50% animals were affected and within 12 hours nearly 100% of the animals were completely paralyzed. These results suggest that folding of recCaspase-3 into its final active conformation is an efficient process and takes relatively very short period of time.

### 10. EXAMPLE

### Split Caspase 9

Caspase-9 is an initiator caspase with a long prodomain that contains a CARD region which is necessary to interact with Apaf-1 in presence of cytochrome C to form a multimeric complex called an apoptosome. Formation of an apoptosome is thought to be a prerequisite for activation of caspase-9. Further, caspase-9 and Apaf-1 are postulated to form a holoenzyme and thus Apaf is required for not only the activation of caspase-9 but also for its activity.

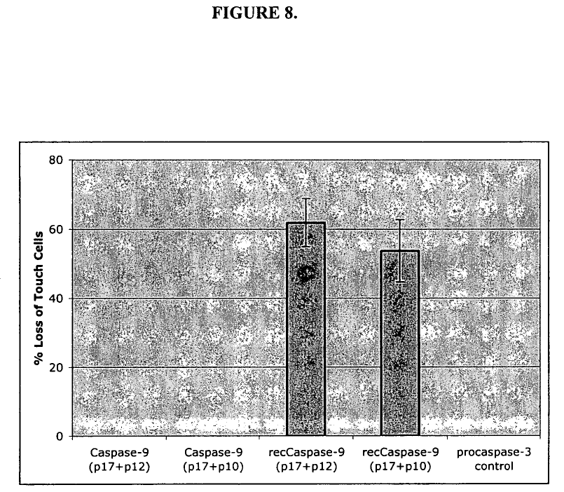

Activated Caspase-9 contains the large subunit p35 (which includes the CARD domain) and the small subunit p12. The small subunit is further processed to p10. For creating recCaspase-9, the CARD domain was removed from the large subunit (resulting in a P17 subunit) and expressed along with either p10 or p12 in C. elegans from the touch cell specific promoter, mec-18. Since activated caspase-9 cleaves procaspase-3, full length procaspase-3 was also included in all experiments. Expression of caspase-9 subunits (large subunit with either p10 or p12) without the leucine zipper sequences did not result in touch cell death (FIG. 8). However, the subunits with leucine zipper sequences resulted in the apoptosis of the touch cells. Apaf-1 may not be required for the activity of caspase-9 but may only play a role in the activation of caspase-9.

### 11. ADDITIONAL REFERENCES

Brockie, P. J., Mellem, J. E., Hills, T., Madsen, D. M., and Maricq, A. V. (2001). The C. elegans glutamate receptor subunit NMR-1 is required for slow NMDA-activated currents that regulate reversal frequency during locomotion. Neuron 31: 617-630.
Chai, J., Shiozaki, E., Srinivasula, S. M., Wu, Q., Datta, P., Alnemri, E. S., and Shi, Y. (2001). Structural basis of caspase-7 inhibition by XIAP. Cell 104: 769-780.
Chalfie, M., Sulston, J. E., White, J. G., Southgate, E., Thomson, J. N., and Brenner, S. (1985). The neural circuit for touch sensitivity in Caenorhabditis elegans. J. Neurosci. 5: 956-964.
Chaudhary, D., O'Rourke, K., Chinnaiyan, A. M., and Dixit, V. M. (1998). The death inhibitory molecules CED-9 and CED-4L use a common mechanism to inhibit the CED-3 death protease. J. Biol. Chem. 273: 17708-17712.
Chinnaiyan, A. M. (1999). The apoptosome: heart and soul of the cell death machine. Neoplasia 1: 5-15.
Chinnaiyan, A. M., Chaudhary, D., O'Rourke, K., Koonin, E. V., and Dixit, V. M. (1997). Role of CED-4 in the activation of CED-3. Nature 388: 728-729.
Cohen, G. M. (1997). Caspases: the executioners of apoptosis. Biochem. J. 326: 1-16.
Ellis, H. M., and Horvitz, H. R. (1986). Genetic control of programmed cell death in the nematode C. elegans. Cell 44: 817-829.
Ghosh, I., Hamilton, A. D., and Regan, L. (2000). Antiparallel leucine zipper-directed protein reassembly: Application to the green fluorescent protein. J. Amer. Chem. Soc. 122: 5658-5659.
Mittl, P. R., Di Marco, S., Krebs, J. F., Bai, X., Karanewsky, D. S., Priestle, J. P., Tomaselli, K. J., and Grutter, M. G. (1997). Structure of recombinant human CPP32 in complex with the tetrapeptide acetyl-Asp-Val-Ala-Asp fluoromethyl ketone. J. Biol. Chem. 272: 6539-6547.
Rotonda, J., Nicholson, D. W., Fazil, K. M., Gallant, M., Gareau, Y., Labelle, M., Peterson, E. P., Rasper, D. M., Ruel, R., Vaillancourt, J. P., et al. (1996). The three-dimensional structure of apopain/CPP32, a key mediator of apoptosis. Nat. Struct. Biol. 3: 619-625.
Salvesen, G. S., and Dixit, V. M. (1999). Caspase activation: the induced-proximity model. Proc. Natl. Acad. Sci. USA 96: 10964-10967.
Shaham, S., and Bargmann, C. I. (2002). Control of neuronal subtype identity by the C. elegans ARID protein CFI-1. Genes Dev. 16: 972-983.
Srinivasula, S. M., Ahmad, M., MacFarlane, M., Luo, Z., Huang, Z., Femandes-Alnemri, T., and Alnemri, E. S. (1998). Generation of constitutively active recombinant caspases-3 and -6 by rearrangement of their subunits. J. Biol. Chem. 273: 10107-10111.
Stanfield, G. M., and Horvitz, H. R. (2000). The ced-8 gene controls the timing of programmed cell deaths in C. elegans. Mol. Cell 5: 423-433.
   Sulston, J. E., and Horvitz, H. R. (1977). Post-embryonic cell lineages of the nematode, Caenorhabditis elegans. Dev. Biol. 56: 110-156.
Wang, S. L., Hawkins, C. J., Yoo, S. J., Muller, H. A., and Hay, B. A. (1999). The Drosophila caspase inhibitor DIAP1 is essential for cell survival and is negatively regulated by HID. Cell 98: 453-463.
Way, J. C., and Chalfie, M. (1989). The mec-3 gene of Caenorhabditis elegans requires its own product for maintained expression and is expressed in three neuronal cell types. Genes Dev. 3: 1823-1833.
Wright, K. M., Linhoff, M. W., Potts, P. R., and Deshmukh, M. (2004). Decreased apoptosome activity with neuronal differentiation sets the threshold for strict IAP regulation of apoptosis. J. Cell Biol. 167: 303-313.
Xue, D., Shaham, S., and Horvitz, H. R. (1996). The Caenorhabditis elegans cell-death protein CED-3 is a cysteine protease with substrate specificities similar to those of the human CPP32 protease. Genes Dev. 10: 1073-1083.
Yang, X., Chang, H. Y., and Baltimore, D. (1998). Essential role of CED-4 oligomerization in CED-3 activation and apoptosis. Science 281: 1355-1357.
Zhang, S., Ma, C., and Chalfie, M. (2004). Combinatorial marking of cells and organelles with reconstituted fluorescent proteins. Cell 119: 137-144.
Brenner, S. (1974). The genetics of Caenorhabditis elegans. Genetics 77: 71-94.
Ellis, H. M., and Horvitz, H. R. (1986). Genetic control of programmed cell death in the nematode C. elegans. Cell 44: 817-829.
Ghosh, I., Hamilton, A. D., and Regan, L. (2000). Antiparallel leucine zipper-directed protein reassembly: Application to the green fluorescent protein. J. Amer. Chem. Soc. 122: 5658-5659.
Mello, C. C., Kramer, J. M., Stinchcomb, D., and Ambros, V. (1991). Efficient gene transfer in C. elegans: extrachromosomal maintenance and integration of transforming sequences. EMBO J. 10: 3959-3970.
O'Hagan, R., Chalfie, M., and Goodman, M. B. (2005). The MEC-4 DEG/ENaC channel of Caenorhabditis elegans touch receptor neurons transduces mechanical signals. Nat. Neurosci. 8: 43-50.
Parrish, J., Li, L., Klotz, K., Ledwich, D., Wang, X., and Xue, D. (2001). Mitochondrial endonuclease G is important for apoptosis in C. elegans. Nature 412: 90-94.
Stanfield, G. M., and Horvitz, H. R. (2000). The ced-8 gene controls the timing of programmed cell deaths in C. elegans. Mol. Cell 5: 423-433.
Sulston, J. E., and Horvitz, H. R. (1977). Post-embryonic cell lineages of the nematode, Caenorhabditis elegans. Dev. Biol. 56: 110-156.
Urlinger, S., Baron, U., Thellmann, M., Hasan, M. T., Bujard, H., and Hillen, W. (2000). Exploring the sequence space for tetracycline-dependent transcriptional activators: novel mutations yield expanded range and sensitivity. Proc. Natl. Acad. Sci. USA 97: 7963-7968.
White, J. G., Southgate, E., Thomson, J. N., and Brenner, S. (1986): The structure of the nervous system of C. elegans. Philos. Trans. R. Soc. Lond. B Biol. Sci. 341, 1-340.

Various publications are cited herein, the contents of which are hereby incorporated by reference in their entireties.

TABLE 2
Effect of ced mutants
on recombinant caspase mediated cell killing activity.

% GFP positive cells in

| Mutant background* | L1 larvae | adults |
|---|---|---|
| recCED-3(uIs37) | — | 5.4 ± 3.5 |
| ced-3(n717):recCED-3(uIs37) | 5.9 ± 1.6 | 5.1 ± 1.5 |
| ced-4(n1162):recCED-3(uIs37) | ND | ND |
| ced-8(n1891):recCED-3(uIs37) | 21.87 ± 1.3 | 4.5 ± 3.5 |
| recCaspase-3(uIs40) | — | 0.9 ± 0.4 |
| ced-3(n717); recCaspase-3(uIs40) | — | 1.0 |

*All strains contained mec-17::gfp (uIs31 or uIs32) in the background

TABLE 3
Promoter combinations
that would label different neuronal groups in C. elegans

| Neuron | Number of | Promoter |
|---|---|---|
| Group^{$} | Neurons | Combination^{#} |
| ADA | 2 | sax-7 + eat-4 |
| ADE | 2 | cat-2 + gpa-14 |
| ADF | 2 | gpa-10 + gpa-13 |
| ADL | 2 | ver-2 |
| AFD | 2 | gcy-8 |
| AIA | 2 | |
| AIB | 2 | odr-2 (2b) + mgl-2 |
| AIM | 2 | zig-3 + cat-1 |
| AIN | 2 | |
| AIY | 2 | ttx-3 + ncs-1 |
| AIZ | 2 | odr-2(2b) + kin-29 |
| ALA | 1 | ceh-14 + ver-3 |
| ALM/PLM/ | 6 | mec-4 |
| AVM/PVM | | |
| ALN/PLN | 4 | lad-2 + unc-53 |
| AQR/PQR | 2 | tax-2 + gcy-36 |
| ASn | 11 | hmr-1 + unc-53 |
| ASE | 2 | gcy-5 or gcy-6 |
| ASG | 2 | tax-2 + lim-6 |
| ASH | 2 | sra-6 + nhr-79 |
| ASI | 2 | gpa-4 |
| ASJ | 2 | tax-2 + gpa-9 |
| ASK | 2 | sra-7 |
| AUA | 2 | eat-4 + ceh-6 |
| AVA | 2 | gpa-14 + flp-18 |
| AVB | 2 | glr-1 + sra-11 |
| AVD | 2 | nmr-1 + cfi-1 |
| AVE | 2 | opt-3 + flp-1 |
| AVF | 2 | |
| AVG | 1 | odr-2 (2b) + nmr-1 |
| AVH | 2 | ceh-6 + ggr-1 |
| AVJ | 2 | |
| AVK | 2 | flp-1 |
| AVL | 1 | |
| AWA | 2 | odr-7 |
| AWB | 2 | str-1 |
| AWC | 2 | odr-1 + tax-4 |
| BAG | 2 | gcy-33 |
| BDU | 2 | ceh-14 + glr-8 |
| CAN | 2 | ceh-23 + ggr-2 |
| CEP | 4 | ace-1 + UL#AL129 |
| DAn | 9 | unc-53 + unc-4 |
| DBn | 7 | vab-7 + unc-5 |
| DDn | 6 | unc-25 + ggr-2 |
| DVA | 1 | zig-5 + nmr-1 |
| DVB | 1 | unc-25 + egl-36 |
| DVC | 1 | ceh-14 + glr-1 |
| FLP | 2 | mec-3 + egl-44 |
| GLR | 6 | |
| HSN | 2 | unc-53 + unc-51 |
| IL1 | 6 | deg-3 + osm-6 |
| IL2 | 6 | oig-1 + osm-3 |
| LUA | 2 | glr-5 + npl-13 |
| OLL | 2 | ace-1 + eat-4 |
| OLQ | 4 | ocr-4 |
| PDA | 1 | dop-2 + itr-1 |
| PDB | 1 | kal-1 + dbl-1 |
| PDE | 2 | gpa-16 + cat-2 |
| PHA | 2 | gcy-12 |
| PHB | 2 | gpa-9 + osm-10 |
| PHC | 2 | dop-1 + ceh-14 |
| PVC | 2 | cfi-1 + deg-3 |
| PVD | 2 | eat-4 + pkc-1 |
| PVN | 2 | |
| PVP | 2 | odr-2(2b) + unc-53 |
| PVQ | 2 | glr-1 + gpa-9 |
| RIC | 2 | |
| RID | 1 | dop-2 + zig-5 |
| RIF | 2 | odr-2(2b) + glr-4 |
| RIG | 2 | glr-1 + flp-18 |
| RIH | 1 | unc-5 + cat-1 |
| RIM | 2 | dop-1 + glr-1 |
| RIP | 2 | |
| RIR | 1 | |
| RIS | 1 | ser-4 + unc-25 |
| RIV | 2 | zig-5 + odr-2 |
| RMD | 6 | rig-5 |
| RME | 4 | lim-4 + unc-47 |
| RMF | 2 | |
| RMG | 2 | avr-15 + goa-1 |
| RMH | 2 | |
| SAA | 4 | lad-2 + unc-42 |
| SAB | 3 | unc-4 + glr-4 |
| SDQ | 2 | pkc-1 + gcy-35 |
| SIA | 4 | lim-4 + sro-1 |
| SIB | 4 | dop-2 + ceh-24 |
| SMB | 4 | |
| SMD | 4 | lad-2 + lim-4 |
| URA | 4 | lim-7 |
| URB | 2 | glr-5 + glr-8 |
| URX | 2 | gpa-8 + pef-1 |
| URY | 4 | glr-4 + tol-1 |
| VAn | 12 | |
| VBn | 11 | pag-3 + acr-5 |
| VCn | 6 | unc-4 + cdh-3 |
| VDn | 13 | unc-55 + unc-14 |
| M1 | 1 | |
| M2 | 1 | tbx-2 + zig-4 |
| M3 | 1 | flp-18 + ceh-2 |
| M4 | 1 | ceh-28 |
| M5 | 1 | tbx-2 + kal-1 |
| I1 | 1 | glr-8 + odr-1 |
| I2 | 1 | glr-8 + npl-8 |
| I3 | 1 | |
| I4 | 1 | |
| I5 | 1 | |
| I6 | 1 | |
| NSM | 1 | cat-1 + glr-7 |
| MI | 1 | ahr-1 + glr-7 |
| MC | 1 | |
| Total | 302 | |

^{$}Neuron groups are based on the classification by White et al.
^{#}Neuron groups that can be labeled by single promoter are indicated in blue, by a combination of two promoters are in black and those that cannot be labeled by two-promoter combination are indicated in red. The data on promoter combinations are extracted from C. elegans expression database (http://wormbase.org/db/searches/expr_search) and (http://chinook.uoregon.edu/promoters.html).

We claim:

1. A nucleic acid comprising a promoter element operably linked to a nucleic acid encoding a split caspase construct comprising a caspase subunit linked to a binder element.

2. The nucleic acid molecule of claim 1, where the binder element comprises a leucine zipper.

3. The nucleic acid molecule of claim 1, where the capsase subunit is a subunit of a caspase selected from the group consisting of caspase-2, caspase-8, caspase-9, caspase-10, caspase-12, caspase-3, caspase-6, caspase-7, caspase-1, caspase-4, caspase-4, caspase-5, caspase-11, and caspase-14.

4. The nucleic acid molecule of claim 2, where the capsase subunit is a subunit of a caspase selected from the group consisting of caspase-2, caspase-8, caspase-9, caspase-10, caspase-12, caspase-3, caspase-6, caspase-7, caspase-1, caspase-4, caspase-4, caspase-5, caspase-11, and caspase-14.

5. The nucleic acid molecule of claim 1, wherein the caspase subunit is a subunit of CED-3.

6. The nucleic acid of claim 1 encoding a first split caspase construct, further comprising a second nucleic acid encoding a second split caspase construct, comprising a second promoter element operably linked to a second nucleic acid encoding a second caspase subunit linked to a second binder element, wherein
the first and second caspase subunits are complementary and together form an active caspase molecule;
the first and second binder elements can form a bond selected from the group consisting of a non-covalent bond and a covalent bond; and
the first and second promoters are not the same.

7. The nucleic acid molecule of 6, where the first and second capsase subunits are subunits of a caspase selected from the group consisting of caspase-2, caspase-8, caspase-9, caspase-10, caspase-12, caspase-3, caspase-6, caspase-7, caspase-1, caspase-4, caspase-4, caspase-5, caspase-11, and caspase-14.

8. A vector containing the nucleic acid molecule of claim 1.

9. A vector containing the nucleic acid of claim 3.

10. A vector containing the nucleic acid of claim 6.

11. A host cell containing the nucleic acid molecule of claim 1.

12. A host cell containing the nucleic acid of claim 3.

13. A host cell containing the nucleic acid of claim 6.

14. A host cell containing the nucleic acid of claim 1 encoding a first caspase subunit, further containing a second nucleic acid encoding a second split caspase construct, comprising a second promoter element operably linked to a second nucleic acid encoding a second caspase subunit linked to a second binder element, wherein
the first and second caspase subunits are complementary and together form an active caspase molecule;
the first and second binder elements can form a bond selected from the group consisting of a non-covalent bond and a covalent bond; and
the first and second promoters are not the same.

15. A non-human transgenic animal containing the host cell of claim 14.

16. A method of detecting coordinate activity of a first and a second promoter element in a host cell containing a first nucleic acid comprising the first promoter operably linked to a nucleic acid encoding a first split caspase construct comprising a first caspase subunit linked to a first binder element and a second nucleic acid comprising the second promoter operably linked to a second nucleic acid encoding a second split caspase construct comprising a second caspase subunit linked to a second binder element, where the first and second split caspase constructs are complementary, the first and second binder elements can form a bond selected from the group consisting of a non-covalent bond and a covalent bond, and the first and second promoters are not the same, comprising detecting the formation of a reconstituted caspase protein from the split caspase constructs by detecting apoptosis.

17. A method of selectively inducing apoptosis in a cell type of interest comprising (i) introducing, into a cell of the cell type of interest, a first nucleic acid comprising a first promoter operably linked to a nucleic acid encoding a first split caspase construct comprising a first caspase subunit linked to a first binder element and a second nucleic acid comprising a second promoter operably linked to a second nucleic acid encoding a second split caspase construct comprising a second caspase subunit linked to a second binder element, where the first and second split caspase constructs are complementary, the first and second binder elements can form a bond selected from the group consisting of a non-covalent bond and a covalent bond, and the first and second promoters are selected such that conditions may be provided so that the first and second promoters are selectively active in the cell type of interest, either constitutively or by induction; and (ii) providing conditions such that the first and second promoters are coordinately active such that the first and second split caspase constructs are coordinately expressed and caspase activity and apoptosis in the cell type of interest are induced.
